(19) United States
(12) Patent Application Publication
(10) Pub. No.: US 2015/0257990 A1
(43) Pub. Date: Sep. 17, 2015

(54) GLITTER AND METHOD FOR PRODUCING SAME

(71) Applicant: Sigmund Lindner GmbH, Warmensteinach (DE)

(72) Inventor: Erwin Pschierer, Kastl (DE)

(21) Appl. No.: 14/656,181

(22) Filed: Mar. 12, 2015

(30) Foreign Application Priority Data
Mar. 14, 2014 (DE) 10 2014 204 819.2

(51) Int. Cl. A61K 8/26; C23C 16/20; A61K 8/02; A61Q 19/00; A61K 8/81; A61K 8/85

(52) U.S. Cl. CPC A61K 8/26; A61K 8/8152; A61K 8/85; A61K 8/0204; A61Q 19/00; C23C 16/20; A61K 2800/10

(57) ABSTRACT

The presently disclosed subject matter relates to glitters having improved surface properties, improved brilliance and improved skinfeel, and also to methods for producing them, the glitters having a coating of a metal, such as aluminium, and comprising a substantially antimony-free, polyester-based foil, and also to methods for producing them.

### RELATED APPLICATIONS

The presently disclosed subject matter claims the benefit of German Patent Application Serial No. 10 2014 204 819.2, filed Mar. 14, 2014; the disclosure of which is incorporated herein by reference in its entirety.

### TECHNICAL FIELD

The presently disclosed subject matter relates to a glitter comprising a substantially antimony-free, polyester-based foil coated with a metal, in some embodiments aluminium, and also to a method for producing said glitter.

### BACKGROUND

Glitters are much employed for producing a glistening surface effect, and have a variety of uses, particularly in cosmetic articles. Such glitters are produced using polymeric foils or films, which are cut by means of a cutting operation into individual, comparably small-sized particles.

One example of a method for producing such glitters is disclosed in DE 102010001971 A1. Disclosed therein are glitters which are coated from all sides.

There continues, however, to be a need for glitters exhibiting improved brilliance. A further desire is to provide glitters having improved skinfeel, i.e. glitters which when employed in cosmetic products evoke a softer and more pleasing sensation on the skin. There is, moreover, a need for glitters which in a coating procedure permit improved, more uniform and smoother coating.

### SUMMARY

The presently disclosed subject matter relates in some embodiments to glitters comprising a polyester-based foil coated with a metal, in some embodiments aluminium, the foil being substantially antimony-free. The presently disclosed subject matter further relates in some embodiments to a method for producing a glitter, in which a polyester-based foil is produced with an antimony-free catalyst, is coated with a metal, in some embodiments aluminium, and the foil is thereafter cut, or the foil is cut and thereafter is coated with a metal, in some embodiments aluminium.

It is an object of the presently disclosed subject matter, accordingly, to provide glitters which exhibit increased brilliance and improved skinfeel. A further object of the presently disclosed subject matter is to provide glitters which in coating allow more uniform and smoother coating and which on account of their high tolerability can be used in cosmetic products. These and other objects are achieved in whole or in part by the presently disclosed subject matter.

Representative embodiments and objects of the presently disclosed subject matter having been mentioned above, further embodiments and objects of the presently disclosed subject matter will become apparent from the detailed description and drawings that follow.

### BRIEF DESCRIPTION OF THE DRAWINGS

Representative embodiments of the drawings will now be described of which:

FIGS. 1 to 4 show diagrammatic sectional views of a glitter of the presently disclosed subject matter with various layer constructions.

FIG. 5 (Example 3) and FIG. 6 (Comparative Example 3) are optical micrographs showing a visual comparison of Example 3 and the comparative example under a temperature load of 230° C. for 5 minutes.

FIG. 7 is a schematic diagram showing criteria for deformation.

### DETAILED DESCRIPTION

The standard approach to producing polyester-based glitters to date has involved the use of an antimony-based catalyst. It has surprisingly emerged that in accordance with the presently disclosed subject matter, providing glitters with improved brilliance and improved skinfeel and also glitters with improved coating characteristics, can be achieved by forgoing such antimony-containing catalysts in the production of polyester-based glitters.

The presently disclosed subject matter relates to a glitter comprising a polyester-based foil coated with a metal, preferably aluminium, where the foil is substantially antimony-free. Substantially antimony-free here means that the foil has an antimony content of less than 100 ppm, preferably less than 50 ppm, more preferably of less than 10 ppm and especially preferably of less than 5 ppm, based on the weight of the foil. The presence of antimony in the foil cannot be ruled out, however. Hence the foil may include antimony as an unavoidable impurity in an amount which is below the limit amount above. According to certain embodiments, moreover, the foil may be transparent or substantially transparent, having a transmissivity for light in the visible range from 380 to 780 nm of 70% or 80% or 90% or more, for example. According to further embodiments, the foil may be coloured or uncoloured, but according to certain embodiments is uncoloured. In certain embodiments, moreover, a colouring layer may be applied to the metal—preferably aluminium—coating or to the foil and the metal coating.

According to certain embodiments, the foil comprises more than 50 wt % of a polyester, based on the weight of the foil. Suitable polyesters in this context include polybutylene terephthalate (PBT), polyethylene terephthalate (PET), and polyethylene isoterephthalate, and also mixtures thereof. In preferred embodiments the foil comprises more than 60 wt % polyester, more preferably more than 70 wt %, more preferably still more than 80 wt % and very preferably more than 90 wt %. In certain embodiments the glitter of the presently disclosed subject matter comprises as its polymer only a polyester-based foil which is substantially antimony-free.

It should be noted here that all percentage figures in the description relate to percent by weight, in the absence of any particular indication.

In addition, the foil may comprise one or more further polymers which can be reacted or mixed with the polyester, in an amount of less than 50 wt %, based on the weight of the foil. Preferably the foil further comprises one or more (meth)acrylate-based polymers in an amount of less than 50 wt %. The expression (meth)acrylate-based polymer here encompasses both acrylate-based polymers and methacrylate-based polymers, and also acrylate- and methacrylate-based (co)polymers, and mixtures thereof. The foil preferably comprises less than 40 wt % of the further polymer, preferably (meth)acrylate-based, more preferably less than 30 wt %, more preferably still less than 20 wt % and very preferably less than 10 wt %. The foil, furthermore, may also comprise foils produced by coextrusion of different polymers, and also foil assemblies composed of different polymeric foils. The presence of further polymer foils alongside the polyester-based foil can therefore not be ruled out, but preferably not more than 10 foils, more preferably not more than 5 foils and very preferably not more than 3 foils in coextruded or foil assembly form are used. In accordance with particular embodiments, however, the glitter itself here is substantially antimony-free, thus having an antimony content of less than 100 ppm, preferably less than 50 ppm, more preferably of less than 10 ppm and especially preferably of less than 5 ppm, based on the weight of the glitter. In preferred embodiments the glitter comprises more than 60 wt % of polyester, more preferably more than 70 wt %, more preferably still more than 80 wt % and very preferably more than 90 wt %. In certain embodiments the glitter of the presently disclosed subject matter comprises as its polymer only polyester that is substantially antimony-free.

According to preferred embodiments, the foil is produced with an antimony-free catalyst. The antimony-free catalyst here preferably comprises at least one metal selected from Ti, Al and Ge. Examples of such catalysts are known from U.K. Thiele, Chemical Fibers International, 54, pp. 162-163, 2004 and U.K. Thiele, International Journal of Polymeric Materials, 50, pp. 387-394, 2001, for example, and include Ecocat-T from Zimmer AG, TG/19 from Teck Cominco, Hombifast from Sachtleben Chemie, Vertec-C400 from Johnson & Matthey, and Tyzor from DuPont, for example.

In a glitter of the presently disclosed subject matter, moreover, the foil is coated further with a metal, preferably aluminium, silver, gold and/or copper, more preferably aluminium. The coating in this case, according to certain embodiments, may be on one side, on two opposite sides, on the entire foil or in some other way. In the glitter, furthermore, in accordance with the presently disclosed subject matter, the metal layer, for example aluminium layer, or the foil and the metal layer may have a coating based on polyurethane, acrylate, styrene-acrylate or epoxy, preferably polyurethane, acrylate or styrene-acrylate, or a coating of a sol-gel-based coating solution. Here as well the coating may be present on one side, two opposite sides, on the entire metal layer, for example aluminium layer, on the foil and on the metal layer, for example aluminium layer, or in some other way. With coated foils of this kind, in particular, it is possible to observe a more uniform and smoother surface of the glitter, this also being reflected, for example, in a further-improved brilliance. For glitters comprising such coated foils, a better skinfeel is obtained as well. It has emerged, moreover, that in the production of such coated glitters, including in particular when using a fluidized bed process, fewer of the glitters adhere, for example, to the walls of the container for their production, resulting in an improved, more uniform coating quality on the part of the glitters. Glitters adhering to the container wall, in contrast, are uncoated or not adequately coated. In the production of such coated glitters, therefore, the resulting production process is also more efficient and better.

The glitter of the presently disclosed subject matter, furthermore, may also comprise other colouring layers and/or effect-imparting layers, of kinds which are known to the skilled person and which a skilled person is able to appropriately apply, from the gas phase or from liquid/solution, for example. In accordance with preferred embodiments, the glitter of the presently disclosed subject matter is not iridescent glitter. Particularly for such iridescent glitters there is no provision for metal coating that may adversely affect the iridescent effect of the glitter, and so the metal coating of the presently disclosed subject matter may be disadvantageous for iridescent glitters. Generally speaking, moreover, iridescent glitters comprise several hundred individual film plies that produce the iris effect, entailing a complicated production procedure. In addition, iridescent glitters are too expensive. Moreover, because of the multiplicity of layers and the lack of metallization, it cannot be ensured that the effect according to the presently disclosed subject matter—improved brilliance and improved skinfeel, and also improved coating characteristics—can be achieved. In particular, the lack of metallization results in less brilliance, and thicker foil thicknesses result in particular in a less pleasant skinfeel.

In accordance with preferred embodiments, the foil that is used in producing the glitter and that is therefore present in the glitter has a thickness of more than 5 μm, preferably more than 10 μm, more preferably of more than 12 μm. In the glitter of the presently disclosed subject matter, in particular with these foils which are thick in comparison to the prior art, it is possible to obtain a further-improved, uniform and smooth surface of the glitter, and an improved brilliance. Furthermore, the thickness is preferably less than 50 μm, for example less than 40 μm, preferably less than 30 μm, more preferably less than 28 μm and very preferably less than 25 μm, including for example less than 20 μm.

Further disclosed is a method for producing a glitter, in which a polyester-based foil is produced with an antimony-free catalyst, is coated with a metal, preferably aluminium, and is thereafter cut, or the foil is cut and thereafter is coated with a metal, preferably aluminium. Preferably the foil is first coated with a metal, preferably aluminium, and the foil thereafter is cut. After the foil has been cut, therefore, particles are produced. The cut form of the foil is not limited, and the particles may be cut, for example, in a hexagon-like form, in other words a form which equates to a hexagon. When particles cut in this way are used, consequently, hexagon-like glitters are produced, although square, round or rectangular glitters or glitters of any other form may also be produced by cutting in a corresponding form. There is no limit on the particle size, which may encompass, for example, a diagonal length (for hexagon-like glitters, for example) or length or a diameter of 50 μm and more, 100 μm and more, or 150 μm and more. For example, moreover, the glitters of the presently disclosed subject matter may have a size (diagonal length, length or diameter or the like) of 500 μm or less. The foil here may have a thickness of more than 5 μm, preferably of more than 10 μm, more preferably of more than 12 μm, and may have the composition stated above. Furthermore, the thickness is preferably less than 50 μm, for example less than 40 μm, preferably less than 30 μm, more preferably less than 28 μm and very preferably less than 25 μm, including for example less than 20 μm. Antimony-free catalysts contemplated are those described above.

The foil in accordance with the presently disclosed subject matter is coated with a metal, preferably aluminium. Coating may take place on one side, on two opposite sides, on the entire foil or in some other way. Methods for the coating of the foils are known to the skilled person. Coating may take place, for example, by vapour deposition, by sprayed application of an aerosol, by painting, or in another way, preferably by vapour deposition. Coating with metal may take place before or after the cutting of the foil. Metallization is preferably accomplished prior to cutting. If the metallization takes place prior to cutting, a polymer coating, based for example on polyurethane, acrylate, styrene-acrylate or epoxy, may be applied in a second coating step, by for example painting, gravure printing, etc. Such polymer coating of the metalized foil facilitates the cutting operation and results in improved production of particles by the cutting operation.

In a method of the presently disclosed subject matter, furthermore, the metal layer, for example an aluminium layer, or the foil and the metal layer, possibly coated with a polymer layer, can be coated, after cutting, with a coating based on polyurethane, acrylate, styrene-acrylate or epoxy. The coating with a polymer based on polyurethane, acrylate, styrene-acrylate or epoxy after the cutting of the foil may take place, for example, by introducing the foil into a polymer solution or by vapour deposition. Coating after cutting with a polymer based on polyurethane, acrylate, styrene-acrylate or epoxy or with a sol-gel-based coating solution is preferably accomplished by a fluidized bed process, as disclosed in DE 102010001971 A1, for example. Suitable coating temperatures here are situated for example at 40 to 140° C., preferably 50 to 100° C., more preferably 60 to 80° C.

For the purpose of the coating of particles, in the case of the fluidized bed process, in principle, the individual particles are coated with a suspension, dispersion or solution. In the case of the fluidized bed process, which is known per se, granules or particles are swirled by a stream of air, the stream of air representing a primary stream, and the coating material envisaged for the particles being introduced via a secondary stream, so that the coating material, which is present in the form of a suspension or dispersion, coats the individual particles completely over the entire outer surface. Fluidized bed processes and apparatus for implementing fluidized bed processes are known, for example, from the published operating instructions for “Mini Glatt mit Microkit”, October 2003, and also from U.S. Pat. No. 3,241,520 and U.S. Pat. No. 5,632,102.

Fluidized bed coating may optionally be repeated in order to produce a multiple coating on the glitters, in order, for example, to obtain modified chemical or physical properties on the part of the coating layer and/or to enable different protective properties.

Additionally disclosed is the use of the glitters of the presently disclosed subject matter in cosmetic products. Cosmetic products in this context include, for example, pastes, ointments, creams, nail varnish, powder, liquid eyeliner, etc., which may comprise the glitters in customary amounts in the formulations. Furthermore, the presently disclosed subject matter also relates to cosmetic products which comprise the glitters of the presently disclosed subject matter.

The presently disclosed subject matter is elucidated in more detail hereinafter with reference to preferred embodiments.

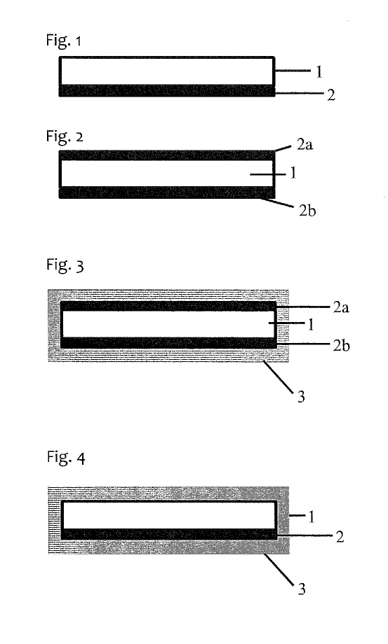

The figures show sectional views through glitters having the construction according to the presently disclosed subject matter. In these views:
FIGS. 1 to 4 show diagrammatic sectional views of a glitter of the presently disclosed subject matter with various layer constructions.

Glitters of the presently disclosed subject matter comprise, for example, glitter particles having a size of 20 μm to 500 μm and a thickness of 25 μm, and consisting, according to a first preferred embodiment, which is shown in FIG. 1, of a transparent, substantially antimony-free polyester foil 1, coated on one side with an aluminium layer 2, and being obtained by cutting of the coated antimony-free polyester foil. A coating of aluminium is applied here by vapour deposition preferably under reduced pressure. According to another embodiment, which is shown in FIG. 2, the substantially antimony-free polyester foil particles 1 may have a coating of aluminium layers 2a, 2b on both sides. A coating of aluminium is applied here by vapour deposition preferably under reduced pressure. Furthermore, the substantially antimony-free polyester foil particles 1, coated on both sides with aluminium layers 2a, 2b, may in a further embodiment, as shown in FIG. 3, additionally have a uniform coating of a layer of polyurethane 3 on all sides. An alternative possibility is for substantially antimony-free polyester foil particles coated on one side with an aluminium layer 2 to have been coated on all sides uniformly with a layer of polyurethane 3 in a further embodiment, as shown in FIG. 4.

In FIGS. 1 to 4, the substantially antimony-free polyester foil in accordance with alternative embodiments may also be coloured, or the glitter particles may be coloured following the application of the metal layer.

The coating with polyurethane 3 in FIGS. 3 and 4 may be produced, for example, by a fluidized bed process.

In this context, generally speaking, the different initial particles are treated under comparable parameters and become fully surrounded by the protective layer material in the fluidized bed process, so that all of the cut edges or fracture edges of the initial particles become enclosed. The precise adaptation of the parameters during the fluidized bed process is guided by the densities and sizes of the respective materials. In FIGS. 3 and 4, all of the outer faces of the particles, with or without aluminium layer, have a surrounding polyurethane layer.

Five kilograms of glitter particles composed of substantially antimony-free polyester and coated with aluminium can be coated with a polyurethane layer, for example, using a WFP-8 fluidized bed unit from DMR Prozesstechnologie GmbH (Switzerland). In this operation, the particles of substantially antimony-free polyester have been produced from a substantially antimony-free polyester foil having a thickness of 25 μm, which has been vapour-coated with aluminium and then coloured red. The particles were cut from this foil in the form, for example, of hexagonal particles with a size of 200 μm, measured over the length of the cut edges parallel to one another. The thickness of the particles is on average 25-35 μm. In the unit stated, these particles were fluidized with an air throughput of 60 m³/h, using process air preheated to 60° C., in order to generate a mixed bed, to which an aqueous polyurethane solution is applied by bottom spraying, i.e. from beneath, for example.

The primary pressure (process air) and the secondary pressure were set in a ratio of 1:2.5, in order to produce effective wetting of the whole of the mixed bed and therefore of the glitter particles present in the mixed bed. The droplets of the polyurethane solution were blown into the fluidized bed uniformly and in fine division. In this example, 1000 ml of polyurethane solution were sprayed in over the course of 60 minutes, in order to counteract rapid incipient drying of the droplets on the particles. At the end of this time, 5 kg of coated glitter were taken from the unit, and the coating was stably crosslinked by subsequent 30-minute heat treatment at 120° C. The result was a homogeneous coating of all of the glitter particles.

Depending on the particle size and particle density of the glitters to be produced, different degrees of fluidization can be set within the fluidized bed. In the case of relatively large glitter particles, depending on the weight of the glitters, a higher process pressure will be necessary than in the case of relatively small particles forming the glitters, in respect both of the process air generating the fluidization and of the polymer solution blown into the fluidized bed. Great expansion of the fluidized bed is necessary in order to ensure a sufficiently great distance between the individual glitter particles, so that the individual glitter particles, accessible separately, can be coated with the protective layer material and at the same time the particles do not stick together during coating.

Instead of a polyurethane solution, the coating material used may also be an aqueous acrylate or styrene-acrylate solution, a sol-gel-based coating solution or an aqueous epoxy solution.

The fluidized bed is generated, for example, by nozzles which produce a primary stream, the pressure of this primary stream determining the length of the spray cone, and by a secondary stream, the pressure of which specifies the width of the spray cone.

It has emerged that the smaller the intended solution droplets or the finer the spray mist intended, the greater the level at which the primary pressure should be set. In the case of relatively small particles, the droplets ought to be extremely fine. Drying of small droplets, however, is quicker than with larger droplets, and so when producing relatively small droplets it is necessary to operate with a greater amount of, for example, aqueous polyurethane solution, so that the moisture level in the fluidized bed is the same.

Alternatively to nozzles arranged in the base (bottom spray), these nozzles may also be provided at another location within the unit, as for example in the upper region of the unit or in the side region (top spray, tangential spray).

The addition of the coating material in the form of polyurethane solutions or other solutions according to the presently disclosed subject matter is made, for example, by a time-controlled metering system, such as a peristaltic pump, for example. Drying or crosslinking is accomplished, depending on the solvent, via the heating of the process air or heating of the mixed bed or fluidized bed. If needed, the glitters can be subsequently subjected to additional thermal processing, in the form of polymerization of the protective layer at temperatures higher than 50° C., for example.

Adaptation in terms of the temperatures may possibly be necessary, and also in terms of the parameters with which the fluidized bed process is carried out.

Coating by employing for example the fluidized bed may optionally be repeated, in order to perform multiple coating of the glitter particles, in order, for example, to produce modified chemical or physical properties of the protective layer and/or to allow different protective properties.

Through the fluidized bed process described above, the multi-layer construction is provided with a coating layer which coats all of the surfaces of the glitter particle.

Depending on the requirements, colour components, in the form of colour pigments, for example, can be introduced either into the coating layer or else beforehand, in conjunction with the application of other layers, in order to colour the surface in question.

The embodiments, refinements and developments above can be combined arbitrarily with one another in so far as makes sense. Further possible refinements, developments and implementations of the presently disclosed subject matter also encompass combinations not explicitly stated of presently disclosed subject matter features described above or hereinafter in relation to the working examples. In particular, the skilled person will also add individual aspects as improvements or additions to the respective basic form of the presently disclosed subject matter.

The presently disclosed subject matter is illustrated below by a number of exemplary embodiments which, however, do not impose any restriction on the presently disclosed subject matter.

Particles of substantially antimony-free polyester have been produced from a substantially antimony-free polyester foil having a thickness of 25 μm, which has been vapour-coated with aluminium and coated with a polymer coating based on acrylate. The particles were cut from this foil in the form of hexagonal particles with sizes of 150 μm (Example 1), 200 μm (Example 2) and 400 μm (Example 3), measured over the length of the cut edges parallel to one another. The thickness of the particles is on average 25-35 μm.

The glitters produced in accordance with Example 3 from antimony-free foil, and a conventional, antimony-containing glitter (PolyFlake, silver ctd., 0.015, Glitterex Corp., USA, Comparative Example 3), were tested for their dimensional stability. Both glitters have a foil thickness of 23 μm to a particle size of 400 μm.

In addition, glitters with particle sizes of 150 μm and 200 μm were investigated for dimensional stability/shape retention under temperature load. In all of the tests, a duplicate determination was carried out.

The shape retention under temperature load is tested by exposing the glitter sample to a defined temperature for a specified time in a heating cabinet.

Test parameters used were as follows:

1. Temperature loading at 175° C. for 15 minutes

2. Temperature loading at 230° C. for 5 minutes

The dimensional stability was determined in this case by employing an optical microscope at a magnification of 25 times up to 200 times.

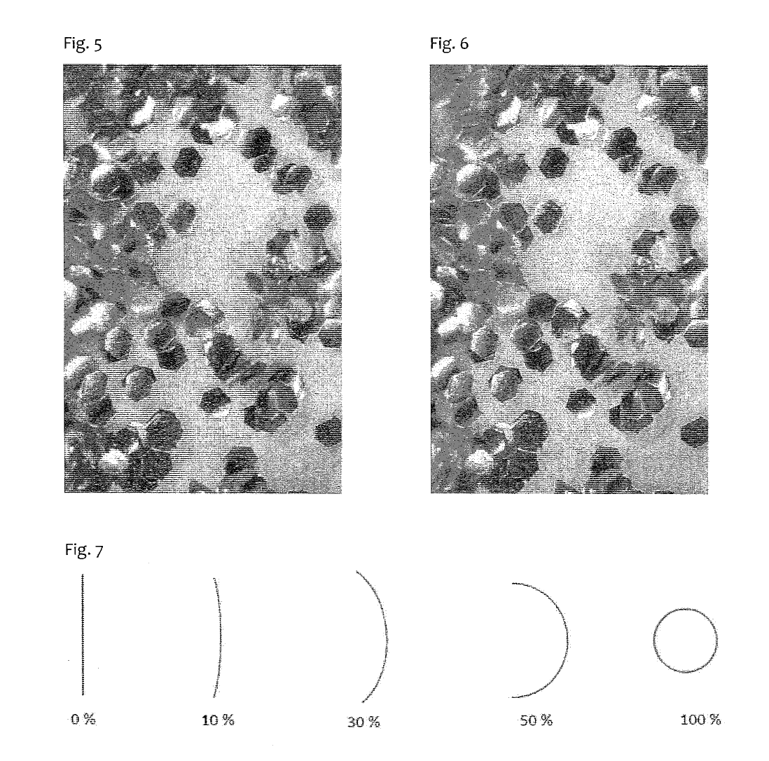

A visual comparison of Example 3 and the comparative example under a temperature load of 230° C. for 5 minutes is apparent from the optical micrographs in FIG. 5 (Example 3) and FIG. 6 (Comparative Example 3). The significant bulging/deformation of the particles in the comparative example is already apparent from these images. It was found that antimony-free glitters are distinguished by better dimensional stability under temperature load. Dimensional stability in this context means that the glitter particles retain their platelet-like, flat form and under temperature load do not sag and do not take on a bulged form.

In a further test to quantify the dimensional stability, glitter particles were exposed to the temperatures and for the times indicated in Table 1.

In order to quantify the dimensional stability, that quality was evaluated empirically on the basis of the optical micrographs, in accordance with the following evaluation scale:

0: no change (deformation 0-10%)

1: slight deformation/acceptable (deformation 10-30%)

2: distinct deformation/unacceptable (deformation 30-50%)

3: loss of shape/unacceptable (deformation >50%)

The criteria for the deformation here can be found in FIG. 7.

TABLE 1
Temperature stability testing results

| | Deformation of glitter | Deformation of glitter |
|---|---|---|
| | from Example 3, | from the comparative |
| | produced free from | example, containing |
| Test parameters | antimony | antimony |
| 175° C./15 minutes | 0 | 1 to 2 |
| 230° C./30 minutes | 1 | 2 |

The most marked effect was found for the size of 400 μm (Example 3), with corresponding results obtained in the other examples as well.

Furthermore, the greater deformation was apparent not only visually, but was also reflected in the tactile perception. The rather three-dimensional character of bulged particles is undesirable in many applications, since it gives the glitters a rougher effect and a non-uniform surface structure. The shape retention is therefore an important quality criterion in respect of the tactile perception as well.

Furthermore, the platelet-like, flat particle morphology is the basis for a mirror-like lustre effect displayed by glitters and therefore for their use as effect material. A bulged particle morphology reduces the mirror effect, thereby causing the desired glitter effect to be less pronounced.

This as well was demonstrated technically by measurement in relation to the brilliance/lightness of the antimony-free-produced glitter, on the basis of colour measurements in the L*a*b* colour space, in which the dL* measurement represents a measure of the lightness. For this a Spectrophotometer CM-3500d, Konica-Minolta, Japan (measurement parameter: measurement geometry d/8°, illuminant D65, normal viewer 10°) is used.

In the measurements, the glitters of Examples 1 and 2 were compared with antimony-containing glitters having sizes of 150 μm (PolyFlake, silver ctd., 0.006, Glitterex Corp., USA, Comparative Example 1) and 200 μm (PolyFlake, silver ctd., 0.008, Glitterex Corp., USA, Comparative Example 2).

With the measurements, positive dL* values were produced in the comparison as shown by Tables 2 and 3 below, which show the measurement results for glitter samples with particle sizes of 200 μm and of 150 μm. This means that in colour terms the antimony-free-produced glitter appears lighter than a comparable antimony-containing glitter, for the same particle size.

TABLE 2
Lightness test results

| | Glitter from | | Difference between |
|---|---|---|---|
| | Comparative | Glitter from | Example 2 and |
| | Example 2 | Example 2 | Comparative Example 2 |
| L* | 58.74 | 64.53 | dL* 5.80 |
| a* | −0.64 | −0.71 | da* −0.05 |
| b* | −1.73 | −0.90 | db* 0.83 |

TABLE 3
Lightness test results

| | Glitter from | | Difference between |
|---|---|---|---|
| | Comparative | Glitter from | Example 1 and |
| | Example 1 | Example 1 | Comparative Example 1 |
| L* | 61.25 | 64.22 | dL* 2.97 |
| a* | −0.66 | −0.92 | da* −0.26 |
| b* | −1.55 | −1.17 | db* 0.38 |

A further advantage is apparent in relation to the temperature stability of the antimony-free-produced glitters.

An investigation was made of the colour change of glitter samples under temperature loading (heating cabinet (120° C./120 minutes)). Table 4 below shows the results for the antimony-free-produced glitter of Example 1 in comparison to two samples of commercial, antimony-containing glitters with a particle size of 150 μm (Comparative Examples 1 and 1′ (1SHB Silver, 0.006, Advance Syntex (P) Ltd., India)).

The value dE*ab here is a measure of the colour difference (colour distance) of two samples—in the present case, the sample before and after temperature loading in each case. The smaller this value, the more alike the two samples are in colour. The antimony-free-produced glitter of Example 1 shows the smallest colour change on temperature loading, with the smallest dE*ab value.

TABLE 4
Colour change under temperature load

| | | Glitter from | Glitter from |
|---|---|---|---|
| | Glitter from | Comparative | Comparative |
| | Example 1 | Example 1 | Example 1′ |
| Colour change | 0.45 | 1.70 | 1.43 |
| dE*ab | | | |

With the antimony-free glitters of the presently disclosed subject matter it is possible to achieve better shape retention under temperature load, better brilliance and better temperature stability, in a manner which could not have been expected. The antimony-free glitters are therefore also available for areas of application which require improved shape retention under temperature load.

It will be understood that various details of the presently disclosed subject matter may be changed without departing from the scope of the presently disclosed subject matter. Furthermore, the foregoing description is for the purpose of illustration only, and not for the purpose of limitation.

What is claimed is:

1. Glitter comprising a polyester-based foil coated with a metal, the foil being substantially antimony-free.

2. Glitter according to claim 1, wherein the metal is aluminium.

3. Glitter according to claim 1, the foil being produced with an antimony-free catalyst.

4. Glitter according to claim 1, the metal layer or the foil and the metal layer having a coating based on polyurethane, acrylate, styrene-acrylate or epoxy or a coating based on a sol-gel-based coating solution.

5. Glitter according to claim 2, the aluminium layer or the foil and the aluminium layer having a coating based on polyurethane, acrylate, styrene-acrylate or epoxy or a coating based on a sol-gel-based coating solution.

6. Glitter according to claim 1, the foil comprising more than 50 wt % of a polyester.

7. Glitter according to claim 1, the foil further comprising a (meth)acrylate-based polymer in an amount of less than 50 wt %.

8. Glitter according to claim 1, the foil having a thickness of more than 5 μm.

9. Glitter according to claim 1, the antimony-free catalyst comprising at least one metal selected from Ti, Al or Ge.

10. Method for producing a glitter, in which a polyester-based foil is produced with an antimony-free catalyst, is coated with a metal, and the foil is thereafter cut, or the foil is cut and thereafter is coated with a metal.

11. The method according to claim 10, wherein the metal is aluminium

12. Method according to claim 10, the foil having a thickness of more than 5 μm.

13. Method according to claim 10, the foil comprising more than 50 wt % of a polyester.

14. Method according to claim 10, the foil further comprising a (meth)acrylate-based polymer in an amount of less than 50 wt %.

15. Method according to claim 10, the antimony-free catalyst comprising at least one metal selected from Ti, Al or Ge.

16. Method according to claim 10, the foil or the metal layer, or the foil and the metal layer having a coating based on polyurethane, acrylate, styrene-acrylate or epoxy or a coating based on a sol-gel-based coating solution.

17. Method according to claim 11, the foil or the aluminium layer, or the foil and the aluminium layer having a coating based on polyurethane, acrylate, styrene-acrylate or epoxy or a coating based on a sol-gel-based coating solution.

18. Use of glitter according to claim 1 in cosmetic products.

19. Cosmetic product comprising a glitter according to claim 1.
